(19) United States
(12) Patent Application Publication
(10) Pub. No.: US 2015/0141951 A1
(43) Pub. Date: May 21, 2015

(54) TARGETED TISSUE HEATING METHODS AND ASSOCIATED SYSTEMS

(71) Applicant: Boston Scientific Scimed, Inc., Maple Grove, MN (US)

(72) Inventors: Sandra NAGALE, Bolton, MA (US); Mark W. BODEN, Harrisville, RI (US); Timothy Paul HARRAH, Cambridge, MA (US); Michael C. LARSON, Colorado Springs, CO (US)

(73) Assignee: Boston Scientific Scimed, Inc.

(21) Appl. No.: 14/546,714

(22) Filed: Nov. 18, 2014

Related U.S. Application Data
(60) Provisional application No. 61/906,246, filed on Nov. 19, 2013.

(51) Int. Cl. A61B 18/08; A61M 31/00; A61F 7/00

(52) U.S. Cl. CPC A61B 18/082; A61F 7/007; A61M 31/00; A61F 2007/0022; A61F 2007/0095; A61B 2018/00791
USPC 604/500; 606/27; 607/113; 607/101; 607/102

(57) ABSTRACT

In one aspect, the present disclosure is directed to a method for treating a bladder within a body of a subject, including inserting one or more heaters into the bladder wall of a subject, and selectively heating the one or more heaters by a remote device.

### CROSS-REFERENCE TO RELATED APPLICATION

This application claims the benefit of U.S. Provisional Application No. 61/906,246, filed Nov. 19, 2013, the disclosure of which is incorporated herein by reference in its entirety.

### FIELD OF THE DISCLOSURE

The disclosure relates generally to targeted tissue heating methods and associated systems.

### BACKGROUND

Overactive Bladder or OAB is one of the factors that can result in urinary incontinence conditions. OAB is a chronic urological condition characterized broadly as the involuntary and uncontrollable urge felt by a subject to relieve the bladder, leading to abnormally high urinating frequency. Such conditions may occur due to frequent and spontaneous contractions of the detrusor muscle of the pelvic region of a subject. Overactive bladders often exhibit localized changes in detrusor morphology, likely originating from defects on cellular and multicellular level. Such cell related deviations may be attributed to local pathological changes in the muscle condition or topology that may contribute to anomalies in the functionality of the detrusor muscle on a macroscopic scale.

Current OAB directed therapies include medication, diet modification, programs in bladder training, electrical stimulation, and surgery. However, existing solutions for OAB, fail to properly address local and anatomical abnormalities of the detrusor muscle, thereby indicating the need for alternative therapies for local bladder abnormalities.

### SUMMARY

In one aspect, the present disclosure is directed to a method for treating a bladder within a body of a subject, including inserting one or more heaters into the bladder wall of a subject, and selectively heating the one or more heaters by a remote device.

In yet another aspect, the present disclosure is directed to a method for treating overactive bladder of a subject, including selectively applying heat to the bladder by heating a plurality of heaters implanted in the bladder wall, and the heat being maintained for a desired time period.

In a further aspect, the present disclosure is directed to a method for treating overactive bladder of a subject, including selectively applying heat to the bladder by heating a plurality of heaters simultaneously, the heaters being implanted in the bladder wall at a depth in the bladder wall sufficient to affect the detrusor muscle of the bladder.

### BRIEF DESCRIPTION OF THE DRAWINGS

The accompanying drawings, which are incorporated in and constitute a part of this specification, illustrate exemplary embodiments of the present disclosure and together with the description, serve to explain principles of the disclosure.

FIG. 1 is a schematic view of an exemplary targeted tissue heating system in accordance with principles of the present disclosure;

FIG. 2 is a schematic view of an exemplary delivery device of the tissue heating system of FIG. 1;

FIG. 3 shows the use of the tissue heating system of FIG. 1 according to one embodiment;

FIG. 4 shows the use of the tissue heating system according to another embodiment of the present disclosure;

FIG. 5 illustrates additional exemplary features of the tissue heating system of FIG. 1; and

FIG. 6 is an exemplary flow chart of a targeted tissue heating method in accordance with principles of the present disclosure.

### DESCRIPTION OF EXEMPLARY EMBODIMENTS

Reference is now made in detail to embodiments of the present disclosure, examples of which are illustrated in the accompanying drawings. Wherever possible, the same reference numbers will be used throughout the drawings to refer to the same or like parts. The term “distal” refers to a position farther away from a user end of the device. The term “proximal” refers a position closer to the user end of the device.

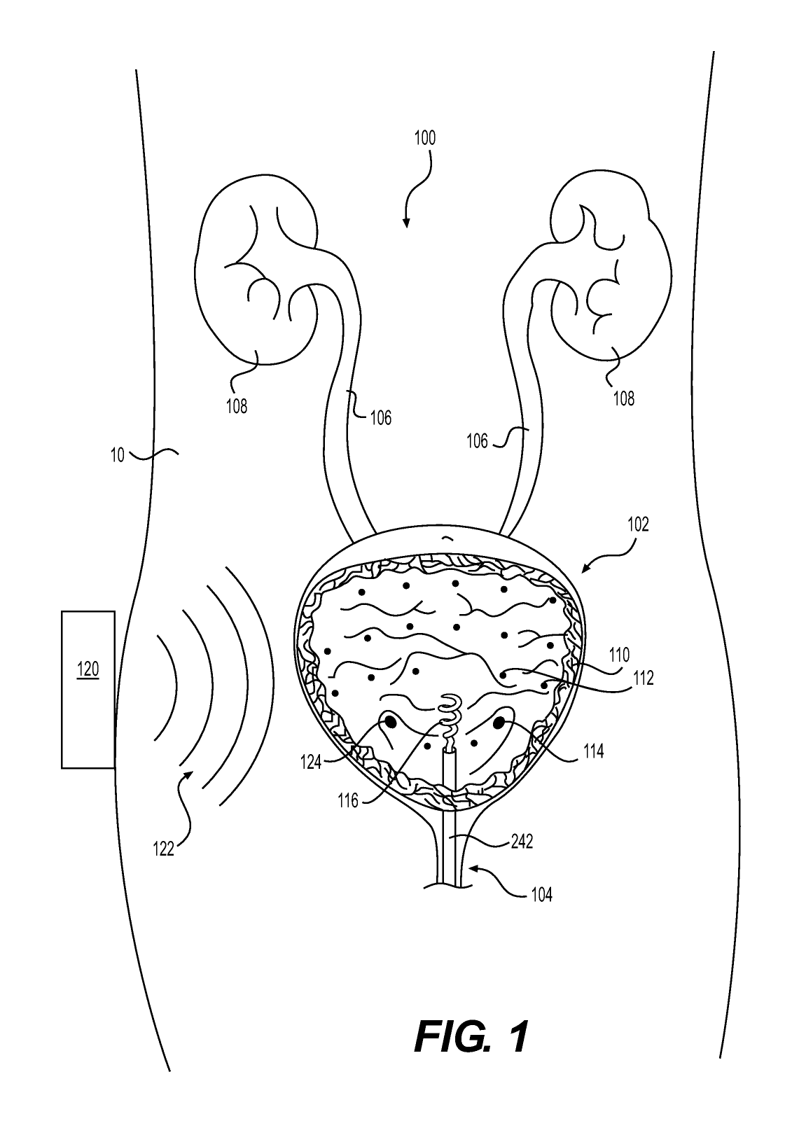

FIG. 1 is a schematic view of an exemplary embodiment of the present disclosure, implemented for and in a urinary tract 100 of a human subject 10. While this disclosure relates to the use of the disclosed system in the urinary tract of a human subject, it is understood that the features of this disclosure could be used in other locations (other organs and tissue) within a human subject. The urinary tract 100 includes, among other structures, a bladder 102 that is in fluid communication with a urethra 104. Bodily fluid, such as urine, travels down from kidneys 108 to the bladder 102 via ureters 106. Muscles (not shown) in the walls of the ureters 106 tighten and relax to force the bodily fluid downward and away from the kidneys 108. The bladder 102 generally accumulates the bodily fluid, which is then discharged from the body through urethra 104. The bladder 102 includes openings 114, 124 for the left ureter and the right ureter, respectively, for receiving bodily fluid from the kidneys 108.

A heater or target may be introduced into the bladder 102 for location within the bladder wall 110 at a depth for heating an area in the mucosal layer and the detrusor muscle of the bladder wall 110. For example, and as will be described in more detail below, the heater may be in the form of a heating element 116 that is embedded into one or more tissue locations 112 in the bladder wall 110. The heating element 116 may be energized wirelessly by signals 122 sent from a signal generating device 120 located outside the body of the subject 10. Energizing the heating element 116 by the external device 120 causes the heating element 116 to increase in temperature and sustain heat in portions of the bladder wall 110. The location of the one or more heater elements 116 within the bladder 102 may be selected according to therapy requirements of the subject 10 suffering from a condition, such as OAB. For example, a plurality of heater elements 116 may be generally evenly distributed within the bladder 102 according to one or more of the tissue locations 112 indicated in FIG. 1.

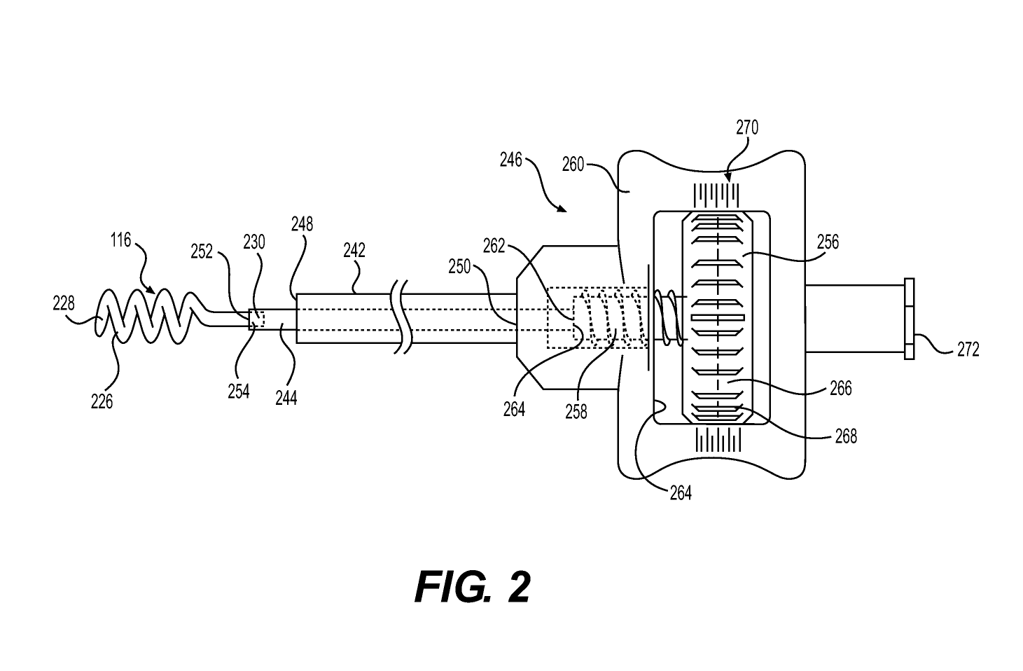

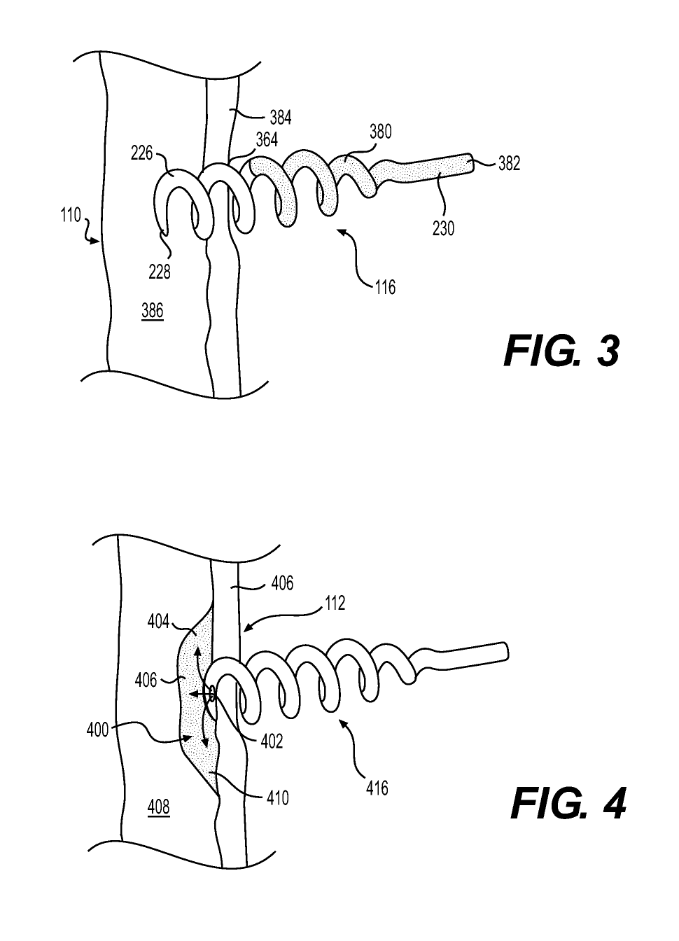

As shown in FIGS. 2 and 3, the heating element 116 may include a distal portion 226 having a helical or coil configuration of a constant and/or varying pitch and extending proximally from a pointed distal tip 228 (e.g., a corkscrew shape). The coil shape may include a lumen size between 1 um and 1 mm. A proximal portion 230 of the heating element 116 may extend proximally from the helical configuration and be generally straight. Other shapes of heating element 116 are possible, for example, distal portion 226 may be provided in any shape that allows for and/or optimizes entry and fixing to the bladder wall 110, and may include an etched, roughened, or barbed portion to assist in fixation. The proximal portion 230 of the heating element 116 may include any number of grooves or protrusions to assist in transferring the requisite forces from a delivery device 240 (FIG. 2) to the heating device 116.

Heating element 116 may be a wire that is fully or partially hollow or solid. The wire may have any cross-section, such as round, flat, square, and/or a combination of such cross-sections. Heating element 116 may be formed of a suitable biocompatible material or materials that are capable of being heated by the signal generating device 120 located outside the body. For example, heating element 116 may be formed from a ferromagnetic material, such as chromium cobalt alloy, stainless steels, iron, nickel and/or cobalt and/or their alloys. Some of such available alloys, characterized by strong ferromagnetism include Co-20Cr-15W-10Ni, Co-20Cr-16Fe-15Ni-7Mo, Co-28Cr-6Mo, Co-35Ni-20Cr-10Mo, or stainless steels: Fe-17Cr-14Ni-2.5Mo, Fe-17Cr-4Ni-4Cu, Fe-18Cr-14Ni-2.5Mo, Fe-19Cr-10Ni, Fe-21Cr-10Ni-3Mn-2.5Mo, Fe-22Cr-13Ni-5Mn-2.5Mo, Fe-23Mn-21Cr-1Mo, Fe-33Pt-18Cr-9Ni-3Mo, Ni-21Cr-9Mo—Nb. Heating element 116 may also be formed from weaker ferromagnetic material such as superparamagnetic iron oxide. Further, as shown in FIG. 3, heating element 116 may include a sleeve or coating 380 to assist in preventing corrosion from fluids such as urine within the bladder 102. The sleeve or coating 380 may cover and extend from a proximal end 382 of the heating element 116 to a location on the distal portion 226 adjacent the depth penetration location 364 of the heating element 116 into the bladder wall 110. Alternatively, the sleeve or coating 380 may be located only on a straight proximal portion of the proximal portion 230 of the heating element 116. The sleeve or coating 380 may include one or more polymers such as, polytetrafluoroethylene (PTFE) and Styrene-block-Isobutylene-block-Styrene (SIBS). Proximal portion 230 may also be fully biodegradable over days or weeks; after the heating element 116 is inserted into the bladder wall, the biodegradable portion falls off (excreted with urine) and the remaining ferromagnetic heating element stays behind inside the bladder wall.

Referring back to FIG. 2, the exemplary medical or surgical delivery device 240 is configured to deliver the heating element 116 into the bladder 102, and to implant the heating element 116 at a desired depth into an internal tissue surface of the bladder wall 110. The delivery device 240 may include an outer tube 242, an inner tube 244 located within the outer tube 242, and an actuator 246 for moving the inner tube 244 proximally and distally with respect to the outer tube 242. Outer tube 242 may include an open distal end 248 and an open proximal end 250 secured to the actuator 246. The outer tube 242 and the inner tube 244 may be formed of flexible hypodermic tubing. Embodiments are intended to include outer and inner tubes 242, 244 of various applicable dimensions, shapes, and materials.

The inner tube 244 may include a distal end 252 with a fitting 254 for receiving the proximal end 230 of heating element 116. The fitting 254 may be a generally cylindrical recess sized to receive a cylindrically shaped proximal end 230 of the heating element 116. The coupling of the fitting 254 and the proximal end 230 of heating element 116 may be a friction fit that allows for the transfer of rotational motion between the inner tube 244 and the heating element 116, while still permitting longitudinal decoupling of inner tube 244 and the heating element 116. Alternatively or additionally, the fitting 254 may include one or more outer or inner surface longitudinal recesses and/or protrusions (not shown) configured to mate with one or more outer or inner surface protrusions and/or recesses (not shown) formed at the proximal end 230 of the heating element 116 so as to assist in the transfer of rotational motion between the inner tube 244 and the heating element 116.

Actuator 246 may be connected to the proximal end 250 of the outer tube 242 and include a knob assembly having a knob 256 configured for imparting rotational and longitudinal movement to the heating element 116. The rotational and longitudinal movement may be achieved by a distal threaded portion 258 of knob 256 that is configured to mate with an inner threaded portion (not shown) of a housing 260 of actuator 246. As shown in dotted lines in FIG. 2, a proximal end 262 of the inner tube 244 may be coupled to a distal end 264 of the threaded portion 258. The knob 256 may be sized and positioned to partially protrude from a window 264 of the housing 260 so that the knob 256 can be more easily gripped for rotation. Further, knob 256 may include a plurality of gripping protrusions 268 and one or more depth indicator markings 266. The depth indicator markings 266 may correspond to one or more depth indicator markings 270 on housing 260. The two sets of depth indicator markings 266, 270 can be configured to indicate the extent of longitudinal movement of the knob 256, and thus the longitudinal movement of inner tube 244 and heating element 116.

With reference to FIGS. 2 and 4, in an alternative embodiment of the present disclosure, the heater can be a heating substance 400 that is injected into the bladder wall 110 at one or more locations 112 (FIG. 1). The heating substance 400 may be supplied through a hollow needle member 416 that is similar in configuration to the heating element 116 of FIGS. 1-3. In contrast to the heating element 116, hollow needle member 416 may be configured to remain coupled to the inner tube 244 during use. In this embodiment, the inner tube 244 may be fluidly coupled to a lumen 402 extending through the hollow needle member 416. Further, a lumen of inner tube 244 may be coupled to a supply tube (not shown) that supplies heating substance 400 to the inner tube 244. For example, knob 256 shown in FIG. 2 may include a lumen extending therethrough that allows for a fluid coupling of a supply tube to the inner tube 244. It is understood that other arrangements are contemplated to convey heating substance 400 to the hollow needle member 416, such as extending the inner tube 244 proximally through a central passage of knob 256 and out a distal end 272 of delivery device 240 to a heating substance supply and injecting mechanism, or merely coupling a needle-type injector through knob 256 and into the lumen of the inner tube 244.

In another embodiment, the hollow needle member 416 may be detached after injection of the heating substance 400. Both the heating substance and the hollow needle member 416 may be heated as disclosed herein.

Heating substance 400 may be a liquid, gas, or gel that includes ferromagnetic particles suspended in a carrier or medium. The ferromagnetic particles may be in the form of small rods, flakes, coils, wavy wires, and/or beads. Further, it is understood that the ferromagnetic particles may or may not be conveyed in a carrier or medium. The size of the particles 404 may vary from tens of micrometers to hundreds of micrometers, larger than the diameter of blood vessels of the subject, to ensure retention within the targeted tissue of the bladder wall. The particles 404 may also be fabricated of similar ferromagnetic materials as described above for ferromagnetic heating elements 116. The particles may be composed of two components: a ferromagnetic component and a biodegradable component and the biodegradable component may be loaded with a drug or protein that is released in the bladder wall over time—weeks or months. The biodegradable component (with or without drug) may coat the ferromagnetic particle or it may be attached to the ferromagnetic particle (for example, a ferromagnetic particle and biodegradable particle are attached via covalent, electrostatic, hydrogen, or van der Waals bonds), Possible drugs may be one or more of anticholinergic, imipramine, desmopressin, estrogens, botulinum toxin, intravesical vanilloids, and lidocaine. The ferromagnetic particles 404 may be suspended in suitable medium 406 such as saline and other physiologic liquids such as hydrogels, such as, Polyethylene glycol-electrolyte (PEG), hyaluronic acid, pluronics, carboxymethylcellulose, alginates, chitosan, poly(lactic) acid, poly(lactic-co-glycolic) acid, glucan gel, Ultra Violet (UV) curable gels, and/or complex polysaccharides (e.g. xanthan gum, guar gum).

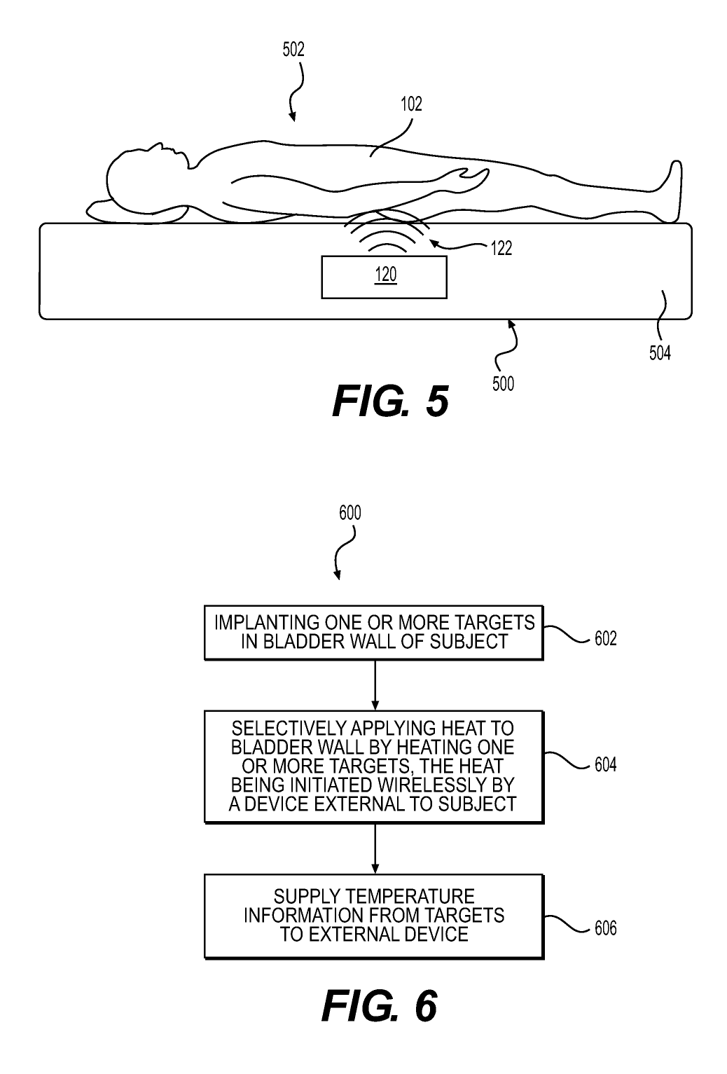

FIG. 5 depicts a selective heat application system 500 for providing targeted tissue heating therapy to the bladder 102 of a subject 502 suffering from OAB. The heat application system 500 may include the signal generating device 120 for sending the wireless signals 122 from a location outside the subject's body 502. The signal generating device 120 may include magnetic or electromagnetic induction coils made from copper or other suitable materials capable of generating magnetic induction fields of substantial intensity and range, when activated. The size of the induction coil depends on the heat desired to be generated by the heaters (116, 400). The induction coil may be either wound in a two-dimensional fashion in the form of a circle (size of a belt buckle) or it may be wound around the belt of the patient. It is understood that multiple induction coils may be used to generate different amounts of heat in distinct spots. For example, as many induction coils as temperature settings exist may be used such that each induction coil may be tuned to all frequencies of all targets at any time. It is understood that some targets may require treatment at higher temperatures due to, for example, abnormalities leading to increased contractions originating at the target. Alternatively, it is understood that some targets may require treatment at lower temperatures. Accordingly, multiple induction coils may be used to individually provide an appropriate temperature for each target.

The external signal generating device 120 may be fed from a high frequency alternating current source (not shown) to generate a highly oscillating electromagnetic field, which will interact wirelessly with the magnetic field of the ferromagnetic heaters embedded within the tissue of the subject. The signal generating device 120 may also include other supporting circuitry components for desired operation. Varying the frequency, phase, and/or the amplitude of the power source or the separation between the external signal generating device 120 and the subject 402 influences the heating therapy parameters.

In another embodiment, external signal generating device 120 may be a Radio Frequency RF transmitter and modulator in wireless communication with RF enabled heaters (116, 400) embedded within bladder wall 102 of subject 402. The embedded heaters (116, 400) may include RF receivers that share a single frequency for reception, or may be tuned at separate frequency channels. When embedded heaters (116, 400) are tuned for same frequency channel, the RF transmitter in the external device, when excited by suitable power source, communicates with RF enabled embedded heaters (116, 400) on the same frequency channel for heating across the embedded locations 112 in the tissue of the subject 502. When different embedded heaters (116, 400) have been tuned for different frequency channels, the RF transmitter and the modulator can be configured to jump from one frequency to another to excite the respective RF enabled embedded heaters (116, 400) for simultaneous heat application therapy.

The external signal generating device 120 may be a hand held device (for example, similar to a hand held ultrasound), or a part of clothing (e.g. a belt buckle), or incorporated in a piece of furniture, such as a bed 504 or chair, to enable wireless heat initiation and control of heat generated in the embedded heaters (116, 400) in the bladder 102 of the subject 502. Incorporating the signal generating device 120 into a piece of furniture allows the subject 502 to receive therapy in a comfortable position and during extended periods of time, e.g., resting or sleeping. The schedule of heat application therapy may also call for incorporation of the external signal generating device 120 into other devices according to the habits and needs of a subject 502. The external signal generating device 120 may also be standalone device that can be placed flat and positioned next to the subject body 502, such as on a belt, or it may be shaped as coil wound around the subject body 502 circumferentially as a belt. The external signal generating device 120 may be located within suitable distances from the subject 502 depending upon the intensity and duration of the heat therapy desired by the subject 502. The heaters (116, 400) may contain conventional temperature sensors such as a resistance temperature detector (RTD sensor), not shown, configured to wirelessly supply the external signal generating device 120 with temperature measurements indicative of the actual temperature supplied by the heaters (116, 400). The temperature measurements can be used by the signal generating device 120 to maintain a desired temperature or to merely monitor the temperature within the subject.

FIG. 6 shows a flow chart 600 of an exemplary method for selective heating of target tissue of a subject using wireless communication. The first step 602 may include implanting one or more heaters or targets in the bladder wall of a subject. As discussed above, in the embodiment shown in FIG. 3 a heater may be in the form of a heating element 116 that is implanted in the subject. In this embodiment, the pointed distal tip 228 of heating element 116 is inserted through a mucosal layer 384 on the surface of a bladder wall 110 and propagates further through in to sub-mucosal layer towards the detrusor muscle 386 of the bladder 102 up to a pre-defined depth within the detrusor muscle 386 of a subject bladder 102, using delivery device 240. The rotational movement of the knob 256 of delivery device 240 translates into a longitudinal movement of the helical shaped heating element 116 into the bladder wall 110. The depth indicator markings 266, 270 on delivery device 240 can assist the user of the device in determining the amount of longitudinal movement of the heating element 116, and thus a depth of penetration into the bladder wall 110.

Also as discussed above, in the embodiment shown in FIG. 4 a heater may be in the form of a heating substance 400 that is injected into the bladder wall 110. The hollow needle member 416 may be inserted through the mucosal layer 406 and further through sub-mucosal layer of the bladder wall 110, up to a desired pre-defined depth between the sub-mucosal layer and detrusor muscle 408 using the rotational movement of the knob 256 of delivery device 240 (FIG. 2) that translates into a longitudinal movement of the hollow needle member 416 into the bladder wall 110. After reaching the desired depth, the heating substance 400 may be injected through the inner tube 244 and lumen 402 of hollow needle member 416 into the bladder tissue between the sub-mucosal layer and the detrusor muscle 408.

The heaters (116, 400) may be provided at multiple locations within the bladder wall 110. For example, the heaters (116, 400) be provided at multiple tissue locations, for example, 10 to 40 sites 112 (FIG. 1) in the bladder wall 110.

In step 604 of FIG. 6, heat is selectively applied to the one or more heaters (116, 400) by the signal generating device 120 in a wireless manner from outside the subject 502. The signal generating device 120 may be located in any appropriate location that would generate signals that would effectuate triggering of the heaters (116, 400). In one embodiment, all of the heaters (116, 400) are actuated simultaneously and generate heat in the range of approximately 41 to 43 degrees Celsius. Alternatively, the heaters (116, 400) could be configured to generate heat at different frequencies and produce varied temperatures. The signal generating device 120 may be configured to generate signals at different frequencies and at different times to selectively actuate different heaters (116, 400) at different times and at different locations within the bladder wall 110. Heat application therapy may be scheduled based on the condition of the subject suffering from, for example, OAB. For example, the heat may be applied according to a preset schedule, or configured to turn on when the bladder 102 has filled to a volume at which contractions usually begin for the subject 502, and the heat discontinued when the bladder has been voided. Further, heat may be applied to different regions of the bladder 102 of a subject at different times of the day. As discussed above, in one embodiment, the signals may be generated over an extended period of time, such as while the subject is sleeping.

In step 606, the heaters (116, 400) may wirelessly supply the external signal generating device 120 with temperature information indicative of the actual temperature supplied by the heaters (116, 400). The temperature measurements can be used by the signal generating device 120 to maintain a desired temperature or to merely monitor the temperature within the subject.

In some embodiments, one or more targets may be heated to a temperature between about 3720 C. to about 70° C. As used herein, the terms “about,” “substantially,” and “approximately,” may indicate a range of values within +/−5% of a stated value.

Embodiments of the present disclosure may be used in various medical, surgical, or non-medical procedures, including any medical procedure where appropriate application of heat or other forms of energy may be desired for target body tissue. The targeted tissue heating for selective and regulated heat application therapy helps to reduce or even treat congenital conditions, such as Overactive Bladder by reducing the number of contractions of the detrusor muscle caused due to local pathological abnormalities. The localized abnormalities such as patchy denervation or increase number of connective tissue between muscles can be reversed or at least stopped from deteriorating further by application of heat at selective tissue intersections to ablate excess tissue formation, and it can also serve to ablate nerve endings to reduce the pathways of communication between the urothelium and the detrusor and nerves. In addition, the method and system detailed out in the different embodiments, in accordance with principles of the disclosure may be implemented to relax various muscles of the subject body to provide relief form tissue tear or rupture. The selective heat application may also address excessive build up of fat for subjects suffering from lifestyles induced ailments, such as, diabetes. The selective heat application may also serve to control peristalsis in the stomach, small and large intestine to slow down or accelerate the contraction and relaxation of muscles to affect the digestive process and feeling of satiety as a possible solution for obese patients.

As discussed above, the targeted tissue heating is achieved, for example, by the heater material, surface area of the heater, the type and characteristics of the energy conveyed by the heater, and the and the constituents of the tissue surrounding the heater.

In addition, aspects of the aforementioned embodiments may be combined with any other aspects of any other embodiments, without departing from the scope of the disclosure.

Other embodiments of the present disclosure will be apparent to those skilled in the art from consideration of the specification and practice of the embodiments disclosed herein. For example, heating element 116 may be configured with a fixed or adjustable stop or marker to help identify the appropriate depth of penetration into the bladder wall 110. Further, consistent with the disclosure above, the disclosed system is not limited to use in the bladder wall 110, but can be used in any other tissue or organ in a subject. For example, the heaters could be used in the stomach or duodenum to reduce contractility and to relax these or other muscles in the body. It is intended that the specification and examples be considered as exemplary only, with a true scope and spirit of the disclosure being indicated by the following claims.

What is claimed is:

1. A method for treating a bladder within a body of a subject, comprising:
inserting one or more heaters into the bladder wall of a subject; and
selectively heating the one or more heaters by a remote device.

2. The method of claim 1, wherein a plurality of heaters are inserted into the bladder wall.

3. The method of claim 2, wherein the plurality of heaters are located at different locations in the bladder wall.

4. The method of claim 3, wherein the different locations include 10 to 40 substantially evenly distributed locations.

5. The method of claim 1, wherein the heating is applied to the bladder wall to heat an area in the mucosal layer and the detrusor muscle.

6. The method of claim 1, wherein the heater includes ferromagnetic material.

7. The method of claim 1, wherein the heater includes a coil shape.

8. The method of claim 7, wherein the heater includes a lumen sized between 1 um and 1 mm.

9. The method of claim 6, wherein the ferromagnetic material is in particle form within a fluid carrier.

10. The method of claim 9, wherein the particle form includes at least one of a rod, flake, coil, wavy wires, or bead.

11. The method of claim 9, wherein the carrier includes at least one of saline or a hydrogel.

12. The method of claim 9, wherein the particle includes a ferromagnetic portion and a biodegradable portion.

13. The method of claim 10, wherein the biodegradable portion includes a drug or a protein that is released over time in the bladder wall.

14. The method of claim 1, wherein the remote device applies RF energy to the one or more heaters.

15. The method of claim 1, wherein the remote device applies alternating current to the one or more heaters.

16. The method of claim 1, wherein the one or more heaters includes a temperature sensor that communicates with the external device.

17. The method of claim 13, wherein the one or more heaters includes at least a first and second heater, and the first and second heaters are configured to selectively apply heat upon receipt of different RF frequencies.

18. The method of claim 1, wherein the external device is removably attached to a body of the subject.

19. A method for treating overactive bladder of a subject, comprising:
selectively applying heat to the bladder by heating a plurality of heaters implanted in the bladder wall; and
the heat being maintained for a desired time period.

20. A method for treating overactive bladder of a subject, comprising:
selectively applying heat to the bladder by heating a plurality of heaters simultaneously, the heaters being implanted in the bladder wall at a depth in the bladder wall sufficient to affect the detrusor muscle of the bladder.
